(19) United States
(12) Patent Application Publication
(10) Pub. No.: US 2017/0276690 A1
(43) Pub. Date: Sep. 28, 2017

(54) Visually-Based Diagnostic Device for Automatic Determination of a Physiologic Level Associated with a Sample

(71) Applicants: Emory University, Atlanta, GA (US); Georgia Tech Research Corporation, Atlanta, GA (US)

(72) Inventors: Wilbur A. Lam, Decatur, GA (US); Erika A. Tyburski, Decatur, GA (US); William A. Stoy, Atlanta, GA (US); Alexander J. Weiss, Fort Washington, PA (US)

(21) Appl. No.: 15/503,936

(22) PCT Filed: Aug. 14, 2015
(86) PCT No.: PCT/US15/45207

§ 371 Date: Feb. 14, 2017

Related U.S. Application Data
(60) Provisional application No. 62/037,166, filed on Aug. 14, 2014.
(60) Provisional application No. 62/182,748, filed on Jun. 22, 2015.

(51) Int. Cl. G01N 33/72; G06T 7/90; B01L 3/00; G06T 7/00; A61B 5/157; A61B 5/15

(52) U.S. Cl. CPC G01N 33/726; A61B 5/157; A61B 5/150022; A61B 5/150343; A61B 5/150351; B01L 3/502; G01N 33/721; G06T 7/0012; G06T 7/90; B01L 2300/042; B01L 2300/0672; B01L 2300/021; B01L 2200/025; B01L 2300/168; G01N 2333/805

(57) ABSTRACT

Diagnostic devices; systems, methods and computer-readable media store instructions related to determining hemoglobin information using those devices. The methods may include processing a captured image of the reservoir and/or device and the device information to determine color information for the solution and the scale, the color information including one or more color attributes; adjusting the color information for the solution based on the color information for the scale; and determining hemoglobin information (e.g., hemoglobin level, disease state and/or calculated hematocrit) based on the adjusted color information and a stored profile information associated with the device information.

### CROSS REFERENCE TO RELATED APPLICATIONS

This application claims priority to Provisional Application Ser. No. 62/037,166 filed on Aug. 14, 2014 and Provisional Application Ser. No. 62/182,748 filed Jun. 22, 2015, which are hereby incorporated by reference in their entireties.

### BACKGROUND

Blood disorders, for example, anemia, or low blood hemoglobin (Hgb) levels, afflict 2 billion people worldwide. However, monitoring such disorders can be costly. Currently, Hgb levels are typically measured from blood samples using hematology analyzers, which are housed in hospitals, clinics, or commercial laboratories, and require skilled technicians to operate.

### SUMMARY

Thus, there is a need for a reliable, inexpensive point-of-care (POC) Hgb test that would enable cost-effective anemia screening and can be used for chronically and acutely anemic patients to self-monitor their disease.

The disclosure relates to diagnostic devices, systems, methods and computer-readable media storing instructions for automatically determining hemoglobin information using the devices.

In some embodiments, the disclosure may relate to a diagnostic, screening or risk-assessment tool/device. In some embodiments, the device may include a cap and a body. The cap may include a plurality of coupling members and a collection member configured for collection of a biological sample (e.g., blood collected from a finger or vein). The plurality of coupling members may surround the collection member. In some embodiments, the body may include a reservoir pre-filled with a solution. The solution may be configured to present a color when it is mixed with the biological sample.

In some embodiments, the disclosure may relate to a method of determining hemoglobin information (e.g., hemoglobin values, hematocrit values, and/or disease state) from a collected biological sample using the diagnostic device according to embodiments. In some embodiments, the methods may include processing a captured image of the device or reservoir and the device information to determine color information for the solution and the scale, the color information including one or more color attributes; adjusting the color information for the solution based on the color information for the scale; and determining a hemoglobin information based on the adjusted color information and a stored profile information associated with the device information. The method may be computer-implemented.

In some embodiments, the disclosure may relate to a computer-readable medium storing instructions for determining hemoglobin information based on a captured image. The medium may be a non-transitory medium.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and descriptions. The components in the figures are not necessarily to scale or shape, emphasis being placed upon illustrating the principles of the disclosure.

FIG. 1 shows an example of a diagnostic device according to embodiments;

FIGS. 2A-D show different views of a diagnostic device according to embodiments;

FIG. 3 shows a system for determining blood hemoglobin levels according to embodiments;

FIG. 4 shows a method of determining blood hemoglobin levels according to embodiments;

FIG. 5 shows a computer system according to embodiments;

FIG. 6 show an example of a diagnostic device according to embodiments;

FIGS. 7A-J show additional views of the diagnostic device according to embodiments; and

FIGS. 8A and B show the diagnostic device with an information member and an enlarged view of the information member, respectively, according to embodiments.

### DESCRIPTION OF THE EMBODIMENTS

The following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

In some embodiments, the disclosure may relate to devices, computer-implemented methods, computer-readable media, and/or systems, that can rapidly and/or accurately determine hemoglobin information, such as quantitative hemoglobin information (e.g., a hemoglobin level (g/dL) and/or a hematocrit level (hct)) and/or a qualitative hemoglobin information (e.g., disease state associated with the hemoglobin and/or hematocrit level) from a collected test sample, such as whole blood. In some embodiments, the devices according to embodiments can be used to collect the test sample for analysis. The devices may be pre-filled with a solution that when mixed with the test sample can result in a color that generally reflects the hemoglobin level. The systems, methods and/or computer-readable media can be used with the device according to embodiments and/or other devices, to rapidly and accurately analyze the test sample to determine hemoglobin information. In some embodiments, the diagnostic analysis system can determine hemoglobin information by calculating color information associated with the mixed sample (also referred to as resultant solution (e.g., a test sample mixed with the pre-filled solution provided in the reservoir)) and correlating the color information to a hemoglobin level or value. The hemoglobin information may correspond to and/or be based on the hemoglobin level. In this way, the methods and/or computer-readable media can account for environmental conditions and human errors in a subjective analysis of the mixed sample.

As used herein, the term “test sample” or “biological sample” generally refers to a material being tested for and/or suspected of containing hemoglobin. The material may include but is not limited to whole blood, blood components (e.g., plasma, red blood cells, platelets, blood serum, etc.), artificial blood product (e.g., blood substitutes, such as recombinant human hemoglobin, cross-linked bovine polyhemoglobin, etc.), as well as any biological material derived from a biological source that may contain or may be suspected of containing hemoglobin. Examples of biological materials may include, but are not limited to, stool, interstitial fluid, saliva, ocular lens fluid, cerebral spinal fluid, sweat, urine, ascites fluid, mucous, nasal fluid, sputum, synovial fluid, peritoneal fluid, vaginal fluid, menses, amniotic fluid, semen, etc.

Diagnostic Device

In some embodiments, the system may include a diagnostic, screening or risk-assessment device configured to collect a test sample for hemoglobin analysis. In some embodiments, the device may include a cap and a body configured to receive the cap. In some embodiments, the cap may be configured to collect a test sample.

In some embodiments, the body may have any shape. In some embodiments, the body may have a circular shape, rectangular shape, as well as other shapes. The body may include a reservoir pre-filled with a solution. In some embodiments, the solution may be filled with a modified TMB and reagent solution. In use, the cap can be placed onto the body so that the test sample can be transferred to the reservoir and mixed with the solution. After the test sample is mixed with the solution (e.g., resultant solution), the test sample can be analyzed.

In some embodiments, the cap may be configured to lock the cap to the body when it is attached so as to prevent the cap from being removed from the body. In some embodiments, the cap may include one or more coupling members configured to mate and/or latch in tension with respective, complimentary coupling members (e.g., tracks, openings, etc.) disposed on the body. In this way, the cap may be configured to snap fit with the body thereby preventing the leakage of the test sample during and/or after the analysis.

In some embodiments, the body may include an information member disposed on an external surface. In some embodiments, the information member may include but are not limited to one or more labels, RFID tags, among others, or a combination thereof. The information member may include (i) one or more legends or scales (also referred to as “color scale” or “standardized scale”) representing the possible colors and associated hemoglobin levels that can result from the mixing a test sample with the solution provided in the reservoir; (ii) an unique identifier identifying the device, color scale, and/or subject from whom the test sample was collected; (iii) one or more legends or scales (also referred to as “reference scale” or “reference legend”) representing one or more colors (e.g., white) configured to correct the capture image for lighting conditions; among others; or a combination thereof. The information member and the corresponding regent solution provided in the reservoir may be customized for the device. For example, the scale and reagent solution may differ for the intended testing population. By way of another example, the information member may include the color scale or standardized scale in addition to a unique identifier identifying the color scale, as well as other information, so that a user can determine a range of hemoglobin values if a diagnostic analysis system according to embodiments is not available at the time of analysis.

In some embodiments, the information member may be disposed on an external surface of the body at a position that is below and/or above the reservoir, a position that at least partially overlaps with the reservoir so as to partially and/or completely surround a region of the reservoir (referred to as “analysis region”), among others or a combination thereof. In some embodiments, the body may include the information member and/or one or more demarcations (e.g., reference point(s)) to at least partially define and/or identify an analysis region, for example, that can be used by a diagnostic analysis system according to embodiments to automatically analyze the hemoglobin value(s) associated with the test sample. The analysis region may refer to a defined area of the body in which at a portion of the reservoir is visible. The analysis may have any shape and/or size. The analysis region is not limited to those shown and described. By way of example, the information member can be used to define and/or identify the analysis region (e.g., a viewing region).

In some embodiments, the demarcations may include but are not limited to one or more labels, color, surface and/or ornamental feature(s) of the body (e.g., recesses or indentations in the body, surface(s), thickness, texture, etc.) , among others, or a combination thereof. The number, size and/or shape of the one or more demarcations and/or information member may correspond to and/or depend on the size and/or shape of the reservoir and/or the body.

In some embodiments, the device may be partially and/or completely transparent. For example, at least the analysis region of the body may be transparent.

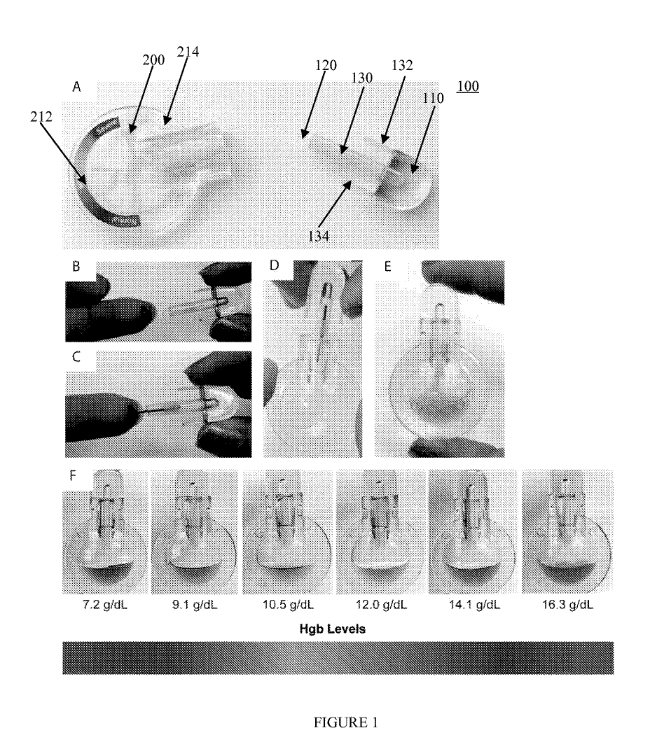

FIGS. 1, 2, and 6-8 show devices according to embodiments. It will be understood that the devices are not limited to the features and/or combination of the features of the body and/or cap shown in the figures. The devices may include any combination of the embodiments of the body and/or cap shown and described with respect to the figures.

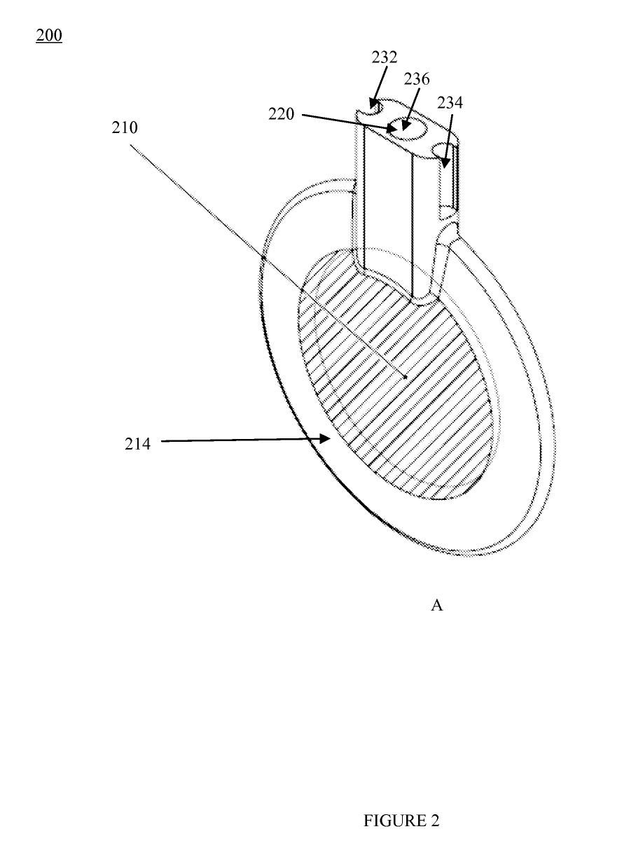

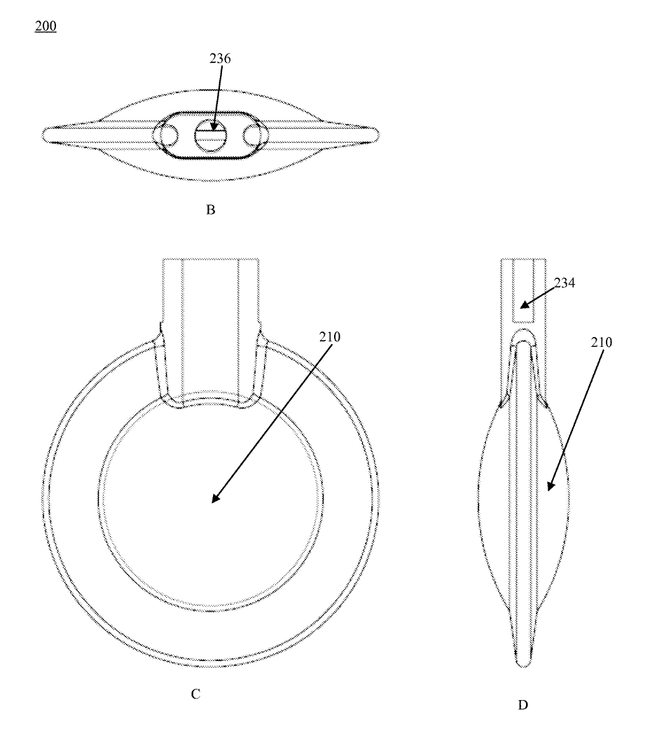

FIGS. 1 and 2 show a diagnostic, screening or risk-assessment device 100 according to embodiments. As shown in the figures, in some embodiments, the diagnostic device 100 may include a cap 110 and a body 200.

In some embodiments, the cap 110 may include a collection member 120 configured to collect the sample. In one embodiment, the cap 110 may include one or more members disposed to encase the collection member 120. In some embodiments, the collection member 120 may be a tube configured to collect the biological sample via capillary action.

FIGS. 1B and 1C show an example of the device 100 being used to analyze the hemoglobin value associated with a whole blood sample, In use, after a finger stick is performed, the collection member 120 included in the cap 110 can directly collect the blood from the finger via capillary action.

In some embodiments, the cap 110 may include one or more coupling members to fixedly attach the cap 110 to the body 200. In some embodiments, the one or more coupling members may protrude from the cap 110. The one or more coupling members may include one or more elongated members configured to engage a complimentary receiving aperture on the body 120, for example, by mating with the aperture and/or latching in tension. In some embodiments, the cap 110 may include two coupling members 132 and 134 may be disposed on the sides of the cap 110. In some embodiments, the cap 110 may include a coupling member 130 that is disposed to surround the collection member 120.

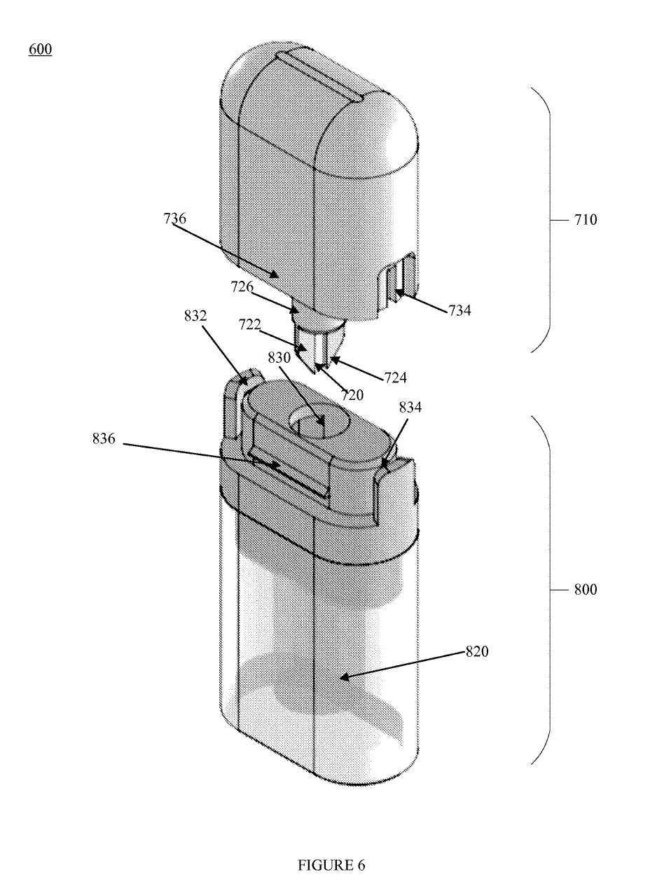

In some embodiments, the body 200 may include a reservoir 210 pre-filled with a reagent solution. FIGS. 2A-2D show different views of the body 200 according to embodiments. As shown in these figures, the reservoir 210 may have a circular shape. In other embodiments, the body 200 and/or reservoir 210 may have a different shape. For example, the reservoir and/or body may have rectangular shape so that the body can be capable of standing. FIGS. 6-8 show a device having a rectangular body.

In some embodiments, the body 200 may include a seal 220 configured to seal the reservoir 210. In some embodiments, the seal 220 may be configured to be broken by the cap 110. In this way, the sterility of the device 100 may be preserved.

In some embodiments, the body 200 may include information member 212 disposed on an external surface. As shown in FIG. 1, the information member 212 may be a label that includes the standardized scale. By way of example, the device 100 may then be used without a diagnostic system according to embodiments. However, it will be understood that the information member may be different. For example, the information member may include printed labels, printed body surfaces, RFID tags and other scale information.

In some embodiments, the information member 212 and the corresponding regent solution provided in the reservoir 210 may be customized for the device. For example, the scale and regent solution may differ for the intended testing population. In some embodiments, the information member 212 may include information identifying the color scale for the device that corresponds to regent, a unique identifier identifying the device (e.g., barcode), among others or a combination thereof. In some embodiments, the information member 212 may be disposed below the reservoir.

In some embodiments, the device 100 may also include one or more demarcations that are disposed on the body 200 to identify and/or define an analysis region. As shown in FIG. 1, the body 200 may include a surface 214 partially surrounds the reservoir 210. The information member 212 may be disposed on the surface 214. By way of example, the surface 214 and/or the information member 212 may identify and/or define the analysis region. In some embodiments, the analysis region may correspond to the reservoir 210. However, it will be understood that the demarcation and analysis region is not limited to as shown and described with respect to body 200 and that other demarcation(s) may be used.

In some embodiments, the body 200 may include one or more coupling members that are complimentary to the coupling members of the cap 110. In some embodiments, the one or more coupling members may include a track, opening, among others, or a combination thereof, for the members 132 and 134. As shown in FIG. 2, the body 200 may include tracks 232 and 234 configured to receive the members 132 and 134 and an opening 230 configured to receive the member 130 and the collection member 120. The tracks 232 and 234 may be disposed on the sides of the body 200, and the opening 230 may be disposed between the tracks 232 and 234.

In use, for example, for testing the hemoglobin level associated with a whole blood sample, as shown in FIGS. 1D and 1E, after the blood is collected in the sample tube 120, the cap 110 can be inserted into the body 200 to break the seal 220. After the cap 110 is fixedly attached to the body 200, the blood sample may react with the reagent in the reservoir 210, thereby causing a change in the color of the pre-filled solution. As shown in FIG. 1F, the resulting color of the solution (from the mixing the blood with the solution) can correspond to a hemoglobin level (range of hemoglobin values). The resulting color can correspond to one of colors included in the scale provided on the device. The user may use their naked eye to interpret the color change and the corresponding hemoglobin level. In this way, the device can provide a simple-to-use, disposable, inexpensive, and is a standalone system that does not require electrical power. Thus, device is feasible as a home-based test for self-screening and/or for simple use in clinical settings.

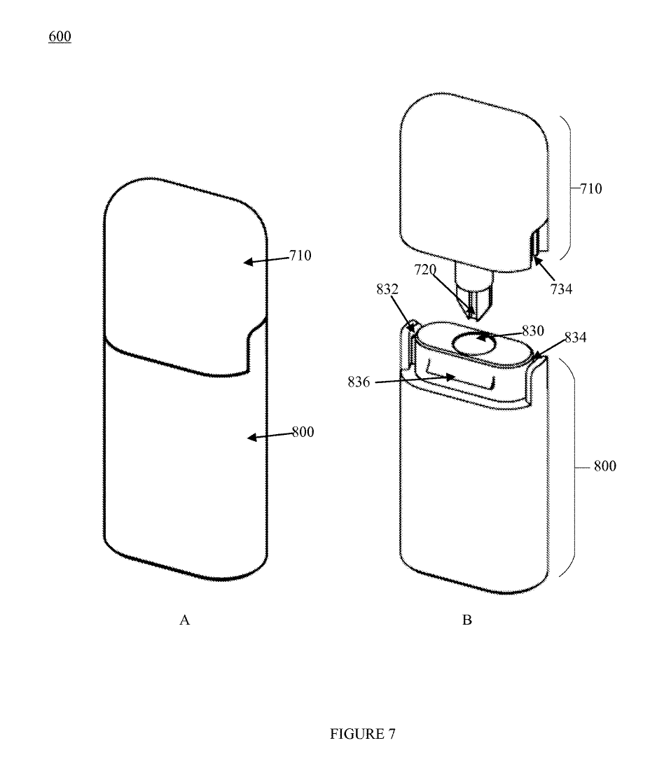

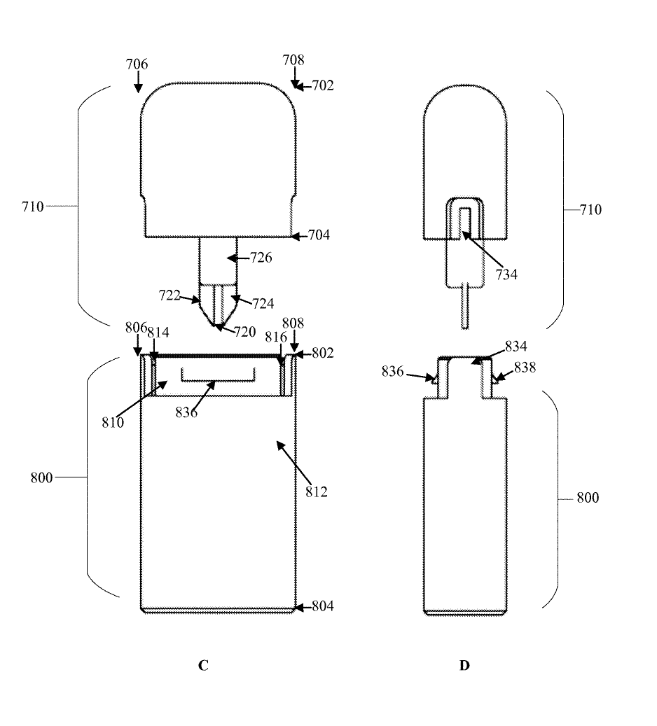

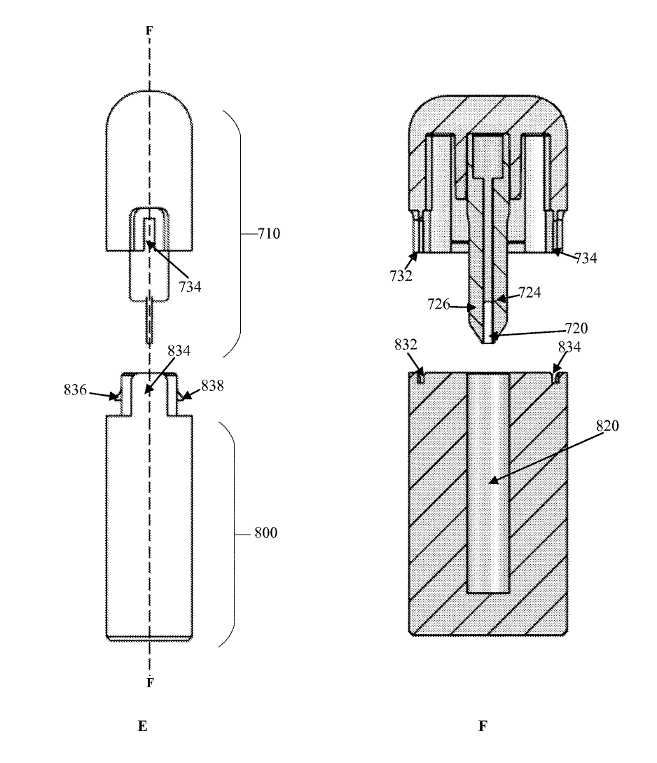

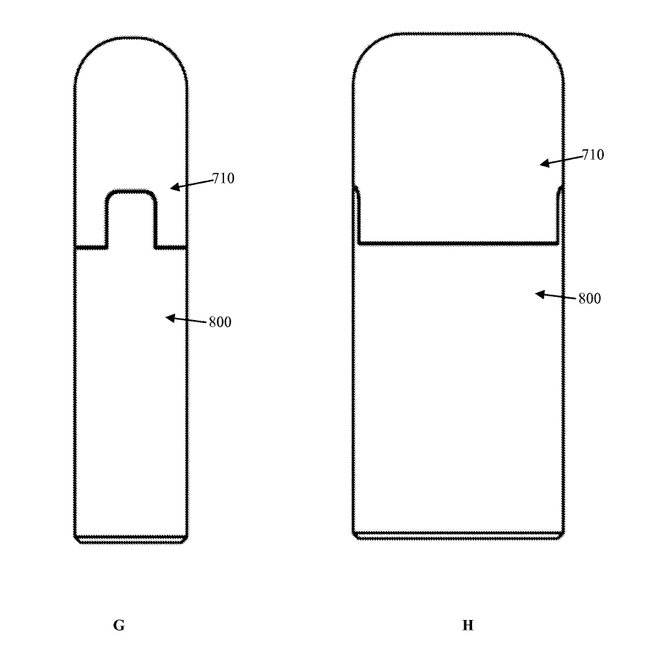

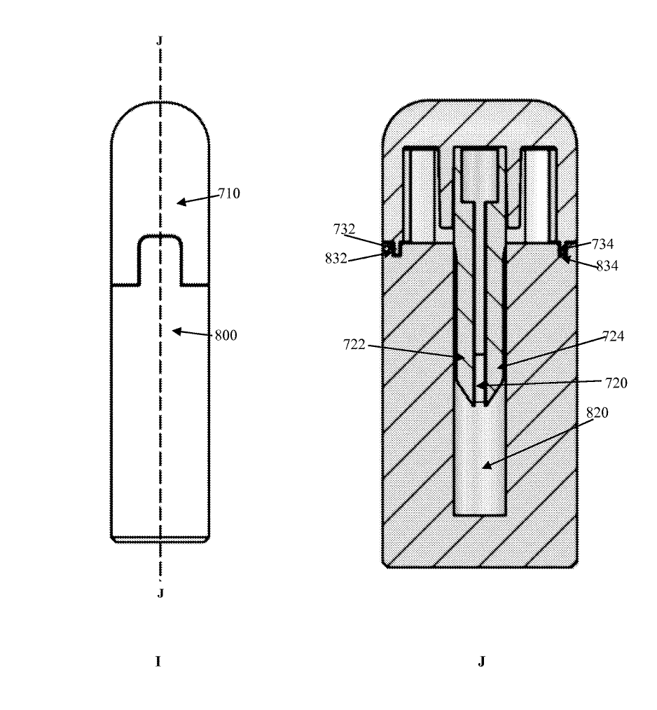

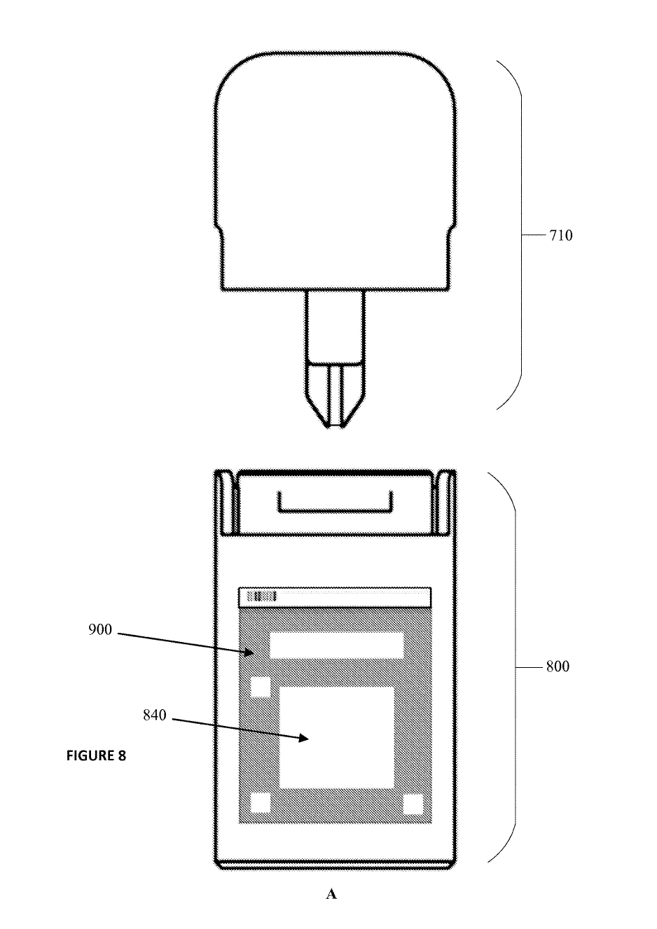

FIGS. 6-8 show a device 600 according to embodiments. Like the device 100, the device 600 may include a cap 710 and a body 800. In some embodiments, the device 600 may have a rectangular shape. FIG. 6 shows a body 800 that is partially transparent. The body shown in FIGS. 7A-J and 8A and B has the same structure but the body 800 is shown opaque so that the mating mechanisms of the body 800 can be more clearly shown. It will be understood that the body 800 shown in FIGS. 7A-J and 8A and B may also be at least partially transparent (e.g., the analysis region), for example, like the body shown in FIG. 6.

In some embodiments, the cap 710 may include a first side (e.g., top side) 702, a second opposing side (bottom side) 704 and a length there between; and a first side 706, a second side 708 and a length there between. In some embodiments, the cap 710 may include a collection member 720 configured to collect the sample. In some embodiments, the collection member 720 may be disposed within the cap 710 and protrude from the second side 704. In some embodiments, the collection member 720 may include an elongated tube (e.g., a pipette or capillary tube). In some embodiments, the cap 710 may be configured to include different sizes of the collection member 720. For example, the size of the collection member 720 may depend on the population to be tested and the amount of blood needed. For example, the collection member 720 may be configured to collect 5 μL and/or 10 μL of blood.

In some embodiments, the cap 710 may include one or more members that at least partially encase the collection member 720. In some embodiments, the cap 710 may include the member 726 that encase a portion of the collection member 720. The member 726 may protrude from the second side 704. In some embodiments, the cap 710 may include one or more members that surround the collection member 720. For example, the cap 710 may include members 722 and 724 that protrude from the member 726 on either side of the collection member 720. In some embodiments, the members 722 and 724 may have a tapered edge configured to break a seal disposed on the reservoir of the body 800.

In some embodiments, the cap 710 may include one or more coupling members to fixedly attach the cap 710 to the body 800. In some embodiments, the one or more coupling members may protrude from the cap 710. The one or more coupling members may include one or more coupling members configured to engage a complimentary member on the body 800, for example, by mating with the aperture and/or latching in tension. In some embodiments, the cap 710 may include two coupling members 732 and 734 disposed on sides 706 and 708 of the cap 710 configured to engage complimentary members disposed on the body 800. The coupling members 732 and 734 may be configured to mate with a complimentary aperture disposed on the body 800. In some embodiments, each of the coupling members 732 and 734 may recessed from the sides 706 and 708 (e.g., outer surface of the cap 710) so that the cap 710 and the body 800 are flush when mated. In some embodiments, the coupling members 732 and 734 may include an aperture between two recessed surfaces.

In some embodiments, the cap 710 may include one or more coupling members 736 and 738 (not shown) disposed in the inner surface between sides 706 and 708. The one or more coupling members 736 and 738 may be a protruding elongated surface configured to engage complimentary members disposed on the body 800. In some embodiments, the members 722 and/or 724 may be configured to assert tension when inserted into complimentary coupling member of the body 800 resulting in the cap 710 latching to the body 800.

In some embodiments, the body 800 may include a first side (e.g., top side) 802, a second opposing side (bottom side) 804 and a length there between; and a first side 806, a second side 808 and a length there between. In some embodiments, the body 800 may include a reservoir 820 pre-filled with a reagent solution. The reservoir 820 may be any size. In some embodiments, the reservoir 820 may be an elongated channel that extends from the first side 802 toward the bottom side 804. The reservoir 820 may have a length that is smaller than the length of the body 800 (between the first side 802 and the second side 804). In some embodiments, the size (e.g., length and/or diameter) of the reservoir 820 may be dependent on the amount of reagent needed for the test. In some embodiments, the body may include a raised surface from the bottom side 804 so as to reduce the surface area within the body 800 for the reservoir 820.

In some embodiments, the body 800 may include an opening 830 disposed on the surface 802 that provides access to the reservoir 820. In some embodiments, like the body 200, the body 800 may include a seal (not shown) configured to seal the reservoir 820. The seal may be fixedly disposed over the opening 830 on the surface 802 so as to completely cover the opening 830 and seal the reservoir 820 and any access thereto. The seal may be made of any material capable of being punctured. In some embodiments, the seal may be configured to be broken by the cap 710 (e.g., the members 722 and 724). In this way, the sterility of the device 800 may be preserved.

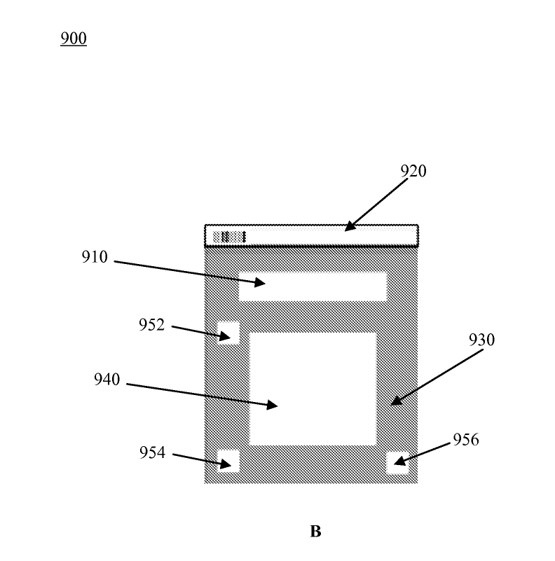

In some embodiments, the device 600 may include information member 900 disposed on an external surface of the body 800, for example, as shown in FIGS. 8A and 8B. In some embodiments, the information member 900 may include one or more labels, RFID tags, among others, or a combination thereof. The information member 900 may include one or more legends or scales 910 that represent the possible colors and associated hemoglobin levels that can result from the mixing a test sample with the solution provided in the reservoir (also referred to as “color scale” or “standardized scale”) and/or that represent one or more colors (e.g., white) configured to correct the capture image for lighting conditions (also referred to as “reference scale” or “reference legend”); (ii) one or more identifiers 920 identifying the device, color scale, collection batch and/or subject from whom the test sample was collected; among others; or a combination thereof. In some embodiments, the information member may be disposed on the body so at least border at least a portion of the reservoir and/or analysis region.

In some embodiments, the information member 900 may demarcate the analysis region 840 of the device 600 by partially and/or completely surrounding a transparent portion of the body 800 with a border 930. In some embodiments, the border 930 may be a colored and/or patterned portion. In some embodiments, the analysis region 840 may correspond to a viewing region 940 of the information member 900. For example, if the information member 900 is a label, the viewing region 940 may be transparent label and/or a cutout (e.g., no label).

In some embodiments, the region 940 and/or 840 may have any size. In some embodiments, the region 940 and/or 840 may have a width substantially corresponds to the circumference of the reservoir 820.

In some embodiments, the device 600 may also include one or more fiducials or reference points disposed on the body 800 to identify the analysis region 840 on the body 800, for example, that can be used by a diagnostic analysis system according to embodiments to automatically analyze the hemoglobin value(s) associated with the test sample. In some embodiments, the one or more demarcations may be disposed on at least one side of the body (e.g., front and/or back surface) surrounding at least a portion of the reservoir (e.g., the analysis region 840). For example, the one or more demarcations may include but are not limited to one or more labels, indentations in the body, other surface or ornamental features (e.g., texturized surface, different thickness, etc.), among others, or a combination thereof. The shape of the one or more demarcations may correspond to the shape of the reservoir and/or body. In some embodiments, the information member and/or one or more demarcations may be disposed on the body so as to partially or completely identify the analysis region (e.g., the viewing region).

In some embodiments, the information member 900 may include reference members 952, 954, and 956 configured to be used by the diagnostic analysis system to automatically determine the viewing window 940 that corresponds to the analysis region 840. In some embodiments, the information member 900 may include more or less reference members (e.g., one reference member, two reference members, and/or four reference members).

In some embodiments, the body 800 may include one or more sections. In some embodiments, the body 800 may include a first section 810 and a second section 812. The first section 810 may be indented from the outer surface and the second section 812 may be flush with the outer surface of the body.

In some embodiments, the body 800 may include one or more coupling members that are complimentary to the coupling members of the cap 710. In some embodiments, the one or more coupling members may include a track, opening, among others, or a combination thereof, for the members 722, 724, 732, 734, 736, and/or 738. The opening 830 may be configured to receive the members 722 and 724 and the collection member 720; and/or protruding elongated members 836 and 838 disposed on sides of the body between the sides 806 and 808 configured to engage the members 736 and 738. The body 800 may also include members 832 and 834 disposed on sides 806 and 808 respectively and openings 814 and 816 disposed between the members 832 and 834 and the first section 810 so as to create tracks that may be configured to respectively receive the members 732 and 734. In this way, the cap 710 may be flush with the body 800 when mated. Also, the reservoir 820 and the opening 830 may be disposed on the body 800 between the one or more coupling members 832, 834, 836, and 838.

In some embodiments, the body 800 may be an integrated molded component. In other embodiments, the body may include one or more layered, molded components. By way of example, the reservoir may be formed in one component and the coupling members may be formed in a second component.

In some embodiments, the device 600 may include a lancet (not shown). In some embodiments, the lancet may be disposed in the cap 710. In some embodiments, the lancet may be separate from the cap 710 and/or the body 800.

In some embodiments, the one, some or all components of the devices may be structured for single use or be disposable. For example, the body and the cap may be configured to be sealed (and not be capable of being easily opened) after the cap is mated with the body. In some embodiments, the kit may also include a lancet. According to some embodiments, a portion or combination of the device and/or lancet may be sold as kit.

It will be understood that a device according to embodiments may be modified based on population, marker levels to be detected, and/or biological sample to be analyzed. The device is not limited to the ranges shown in FIGS. 1A-F. For example, the pre-filled solution and reagent may be altered for a different range of hemoglobin levels. In other example, a device may be used to analyze biomarkers in for example, a different biological sample, such as urine.

Diagnostic Analysis System

In some embodiments, a device according to embodiments may be used with a diagnostic analysis system, for example, a computer program downloaded on a user device to provide a more accurate, rapid, and automatic determination of the hemoglobin level associated with a test sample and that can also be used for data transmission. By using the program, an accurate hemoglobin value can be determined rather than a range of values of hemoglobin determined by using the device alone with a color scale. Additionally, a program may eliminate user subjectivity and errors reading the color and take into account the lighting conditions.

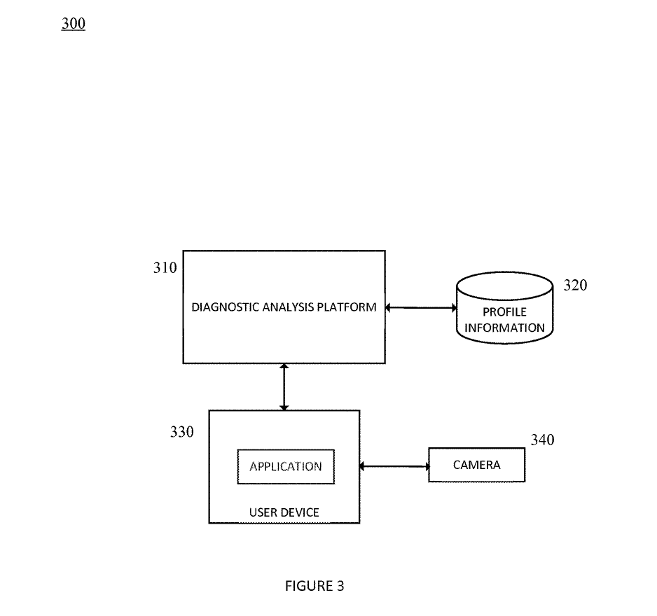

FIG. 3 shows an example of a system 300 capable of automatically and accurately determining hemoglobin information using a device according to embodiments. The hemoglobin information may include but is not limited to a quantitative and/or qualitative hemoglobin value. For example, a quantitative hemoglobin value may include a hemoglobin and/or hematocrit value (e.g., approximately three times the hemoglobin value). The qualitative hemoglobin value may include a disease state associated with the hemoglobin and/or hematocrit value. For example, the disease state may be a scaled disease state including but not limited to normal, moderately anemic, severely anemic, etc. It will also be understood that the system 300 may be used with other devices.

The system 300 may include any number of modules that communicate with other through electrical or data connections (not shown). In some embodiments, the modules may be connected via a wired network, wireless network, or combination thereof. In some embodiments, the networks may be encrypted. In some embodiments, the wired network may be, but is not limited to, a local area network, such as Ethernet, or wide area network. In some embodiments, the wireless network may be, but is not limited to, any one of a wireless wide area network, a wireless local area network, a Bluetooth network, a radio frequency network, or another similarly functioning wireless network.

Although the modules of the system are shown as being directly connected, the modules may be indirectly connected to one or more of the other modules of the system. In some embodiments, a module may be only directly connected to one or more of the other modules of the system.

In some embodiments, the modules and/or systems of the system 300 may be connected to a data network, a wireless network, or any combination thereof. In some embodiments, any of the modules and/or systems of the system 300 may be at least in part be based on cloud computing architecture. In some embodiments, the modules and/or systems may be applied to a self-hosted private cloud based architecture, a dedicated public cloud, a partner-hosted private cloud, as well as any cloud based computing architecture.

As shown in FIG. 3, the system 300 may include a diagnostic analysis platform 310 configured to determine the hemoglobin information as well as other marker levels associated with a test sample. The platform 310 may be configured to factor the testing conditions (e.g., ambient light conditions) in determining the hemoglobin information and/or other marker level.

In some embodiments, the system 300 may include a profile information database 320 that may include one or more marker profiles. For example, the database 320 may include profile information for each scale associated with the diagnostic device. The profile information may include but is not limited to a standardized scale based on control samples for the one or more solutions disposed in the device, size of the device (e.g., amount of sample to be collected), and/or population to be tested. As mentioned above, there may be more than one scales and corresponding solutions for different populations.

In some embodiments, the system 300 may include a user device 330 for use with the platform 310. The user device 330 can be any type of computing device, for example, configured to communicate to a network. For example, the user device 330 may be a mobile communication device, such a smart phone or a table, personal computer, or a super computer, having an application loaded and running on the user device 330. FIG. 5 shows an example of a user device 500 according to some embodiments. The user device 330 may include or may communicate with a camera 340. The application may be capable of performing all or some of the functions of the platform 310.

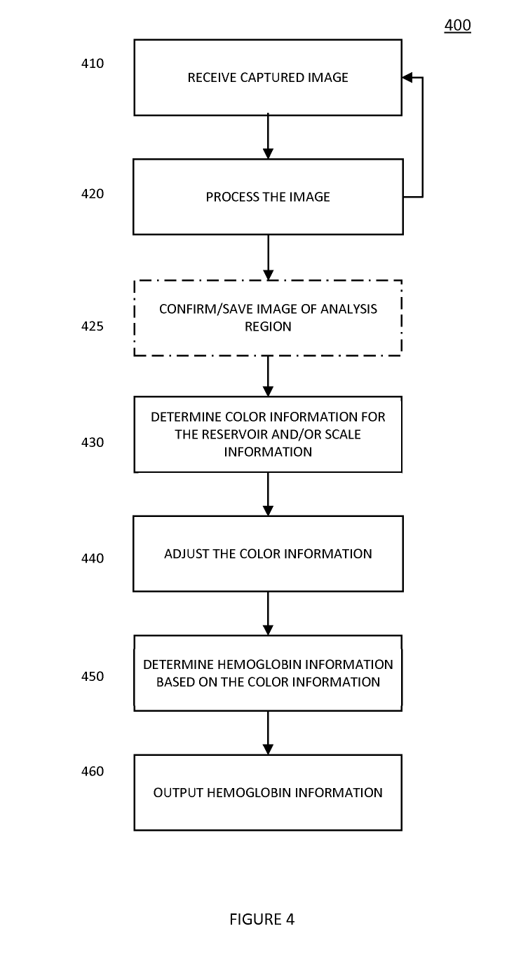

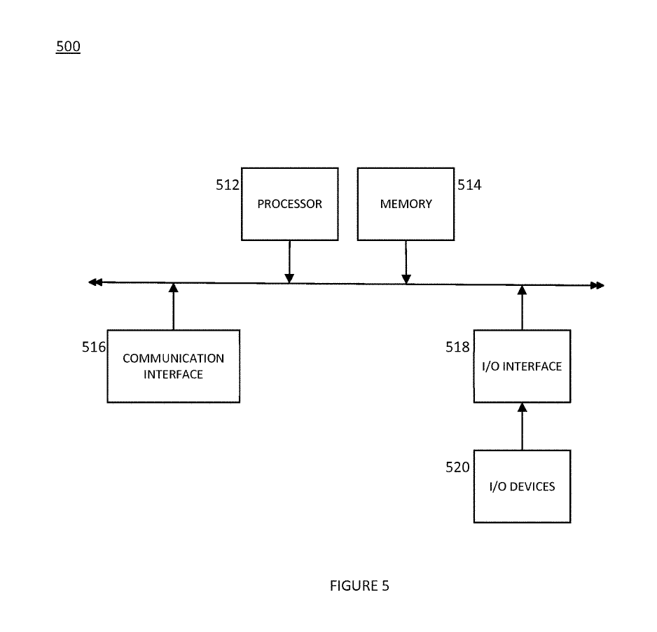

FIG. 4 illustrates a method 400 for processing aligned sequence information to generate one or more consensus sequences. The system for carrying out the embodiments of the methods disclosed herein is not limited to the system shown in FIG. 3. Other systems may be used.

The methods of the disclosure are not limited to the steps described herein. The steps may be individually modified or omitted, as well as additional steps may be added. It will be also understood that at least some of the steps may be performed in parallel.

Unless stated otherwise as apparent from the following discussion, it will be appreciated that terms such as “capturing,” comparing,” “generating,” “determining,” “obtaining,” “processing,” “computing,” “selecting,” “receiving,” “correcting,” “estimating,” “calculating,” “quantifying,” “causing,” “confirming,” “outputting,” “acquiring,” “analyzing,” “approximating,” “continuing,” “resuming,” “using,” “retrieving,” “performing,” “adjusting,” “calibrating,” “reviewing,” “correlating,' or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices.

As shown in FIG. 4, the method 400 may include a step 410 of receiving a captured image. The captured image may include the body of the device (e.g., reservoir and the information member (e.g., reference scale and/or device identifier) (e.g., analysis region 840,940 and information member 900)). In some embodiments, after the user opens the application, the application can be programmed to automatically utilize the camera on the user device to cause the camera to capture the image of the device automatically.

In some embodiments, the method 400 may include a step 420 of processing the captured image to determine the analysis region and/or to determine the image quality. In some embodiments, the platform 300 may automatically determine the analysis region (e.g., analysis region 840/940). By way of example, the platform 300 may use visual analysis to locate the reference members 952, 954 and/or 956 and/or the information member 900 on the face of device to determine the analysis region 840/940. For example, the platform 300 may use edge detection to identify the reference members 952, 954 and/or 956 and/or the information member 900 printed/stickered on the device face.

By way of another example, in some embodiments, the processing step 420 may include converting the captured image to black and white using a threshold to differentiate the analysis region from the rest of the body in the image. For example, the analysis region may be white and the border (e.g., demarcation(s) and rest of body) may be black for locating the analysis region.

In some embodiments, the processing step 420 may optionally include standardizing spatial orientation of the captured image. In some embodiments, the platform 300 can correct the analysis region (e.g., 840/940) for orientation and/or distance from the camera using, for example, the one or more reference members (e.g., 952, 954 and/or 956). In this way, to the platform 300 can zoom in on resultant solution and device face with the scale information (e.g., 910).

In some embodiments, the processing step 420 may optionally include determining image quality based on determining the analysis region. If the platform 300 cannot determine the analysis region or determines that the captured image is of insufficient quality, the processing step 420 may further include causing a request (for example, a prompt) to be transmitted to a user indicating a recapture of the image is necessary to ensure that the entire device face and region of interest are captured at a sufficient quality and/or include additional instructions for better image quality (e.g., suggest a distance for the camera from the device).

In some embodiments, the method 400 may optionally include a step 425 of requesting confirmation of the image. In some embodiments, the method 400 may transmit a request that the determined analysis region is correct, for example, by requesting a user to tap the analysis region on a processed image. In other embodiments, the step 425 may be omitted.

In some embodiments, the platform 300 may store the image captured and/or processed after steps 420 and/or 425. For example, the platform 300 may store the (captured) image and/or processed (captured) image with the unique identifier for the device and/or subject (from which the biological sample was obtained) included with the information member.

In some embodiments, the method 400 may include a step 430 of determining color information for the analysis region and/or the identification member (e.g., reference scale). In some embodiments, the color information may include one or more color attributes. The one or more color attributes may relate to quantifiable metrics associated with a colorimeter. By way of example, one or more color attributes may include but is not limited to intensity, brightness, hue in RGB and/or XY, among others, or a combination thereof. In some embodiments, the step 430 may determine the color attributes associated with the analysis region and the color attributes associated with the identification member (e.g., reference scale (portion of white)) provided on the device. For example, the step 430 may determine one or more color attributes for the analysis region and the scale, for example, by averaging the one or more color attributes for the pixels located in the analysis region and scale, respectively. In some embodiments, the step 430 may determine the color attributes using the color scale legend provided on the device and/or stored profile associated with the device.

In some embodiments, the method 400 may include a step 440 of adjusting the color information for analysis region based on the color information determined for the scale information (e.g., color scale and/or reference scale). By way of example, the step 440 may use the color information for the white reference point (reference scale) provided on the identification member included in the image to correct the color information for the analysis region. In this way, the platform 300 may correct the color information for the analysis region for different ambient lighting situations. By way of example, the platform 300 may multiply, divide, add or subtract from the color information for the analysis region (e.g., solution mixed with the test sample) based on the color information for the reference scale. The platform 300 may thereby provide a more accurate determination of the hemoglobin level than human eye analysis.

In some embodiments, the method 400 may include a step 450 of determining the hemoglobin information based on the corrected color information for the solution. In some embodiments, the step 450 may include retrieving the profile associated with the solution provided in the device based on the identification information, for example, from the profile information database 320. In some embodiments, the profile may be a plane in a three-dimensional space that correlates hemoglobin estimation levels to quantified color values. In some embodiments, the step 450 may compare the corrected color information determined for the analysis region to the corresponding stored profile in the profile information database 320. By way of example, for hemoglobin values, the step 450 may determine the hemoglobin value by correlating the corrected quantified color information for the resultant solution provided in the analysis region to a quantitative hemoglobin value in the corresponding stored profile. In some embodiments, the step 450 may include determining the hemoglobin value by correlating the corrected color information for the analysis region to a hemoglobin value on a plane in a three-dimension space.

In some embodiments, the step 450 may include determining additional quantitative hemoglobin information. For example, the step 450 may include determining the hematocrit value by multiplying the determined hemoglobin value by 3.

In other embodiments, the step 450 may be performed step 440 so that the color attributes are corrected after the hemoglobin information is determined.

In some embodiments, the step 450 may also include determining qualitative hemoglobin information. For example, the step 450 may include determining the disease state associated with the hemoglobin value and/or hematocrit value. The disease state associated with different ranges of hemoglobin levels may be stored with the profile.

In some embodiments, the method 400 may include a step 460 of outputting the hemoglobin information (e.g., hemoglobin value, hematocrit value, and/or disease state). In some embodiments, the step 460 may include displaying the hemoglobin information; storing the hemoglobin information, for example with the patient and/or device identifier; outputting the hemoglobin for further analysis and/or storage; among others; or a combination thereof. In some embodiments, the outputted hemoglobin and/or hematocrit value may be rounded to the tenths place.

FIG. 5 is a block diagram showing a computer system 500. The modules of the computer system 500 may be included in at least some of the platforms, system and/or modules, as well as other devices of system 300.

The systems may include any number of modules that communicate with other through electrical or data connections (not shown). In some embodiments, the modules may be connected via a wired network, wireless network, or combination thereof. In some embodiments, the networks may be encrypted. In some embodiments, the wired network may be, but is not limited to, a local area network, such as Ethernet, or wide area network. In some embodiments, the wireless network may be, but is not limited to, any one of a wireless wide area network, a wireless local area network, a Bluetooth network, a radiofrequency network, or another similarly functioning wireless network.

It is also to be understood that the systems may omit any of the modules illustrated and/or may include additional modules not shown. It is also be understood that more than one module may be part of the system although one of each module is illustrated in the system. It is further to be understood that each of the plurality of modules may be different or may be the same. It is also to be understood that the modules may omit any of the components illustrated and/or may include additional component(s) not shown.

In some embodiments, the modules provided within the systems may be time synchronized. In further embodiments, the systems may be time synchronized with other systems, such as those systems that may be on the medical facility network.

The system 500 may be a computing system, such as a workstation, computer, or the like. The system 500 may include one or more processors 512. The processor(s) 512 (also referred to as central processing units, or CPUs) may be any known central processing unit, a processor, or a microprocessor. The CPU 512 may be coupled directly or indirectly to one or more computer—readable storage media (e.g., memory) 514. The memory 514 may include random access memory (RAM), read only memory (ROM), disk drive, tape drive, etc., or a combinations thereof. The memory 514 may be configured to store programs and data, including data structures. In some embodiments, the memory 514 may also include a frame buffer for storing data arrays.

In some embodiments, another computer system may assume the data analysis or other functions of the CPU 512. In response to commands received from an input device, the programs or data stored in the memory 514 may be archived in long term storage or may be further processed by the processor and presented on a display.

In some embodiments, the system 510 may include a communication interface 516 configured to conduct receiving and transmitting of data between other modules on the system and/or network. The communication interface 516 may be a wired and/or wireless interface, a switched circuit wireless interface, a network of data processing devices, such as LAN, WAN, the internet, or combination thereof. The communication interface may be configured to execute various communication protocols, such as Bluetooth, wireless, and Ethernet, in order to establish and maintain communication with at least another module on the network.

In some embodiments, the system 510 may include an input/output interface 518 configured for receiving information from one or more input devices 520 (e.g., a keyboard, a mouse, camera, and the like) and/or conveying information to one or more output devices 520 (e.g., a printer, a CD writer, a DVD writer, portable flash memory, etc.). In some embodiments, the one or more input devices 520 may configured to control, for example, the generation of the consensus sequence(s), the display of the consensus sequence(s) on a display, the printing of the consensus sequence(s) by a printer interface, among other things.

It is to be understood that the embodiments of the disclosure may be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine comprising any suitable architecture. The system and method of the present disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

In some embodiments, the disclosed methods (e.g., FIG. 4) may be implemented using software applications that are stored in a memory and executed by a processor (e.g., CPU) provided on the system 300. In some embodiments, the disclosed methods may be implanted using software applications that are stored in memories and executed by CPUs distributed across the system 300. As such, any of the systems and/or modules of the system 300 may be a general purpose computer system, such as system 500, that becomes a specific purpose computer system when executing the routine of the disclosure. The systems and/or modules of the system 300 may also include an operating system and micro instruction code. The various processes and functions described herein may either be part of the micro instruction code or part of the application program or routine (or combination thereof) that is executed via the operating system.

If written in a programming language conforming to a recognized standard, sequences of instructions designed to implement the methods may be compiled for execution on a variety of hardware platforms and for interface to a variety of operating systems. In addition, embodiments are not described with reference to any particular programming language. It will be appreciated that a variety of programming languages may be used to implement embodiments of the disclosure. An example of hardware for performing the described functions is shown in FIGS. 3 and 5.

It is to be further understood that, because some of the constituent system components and method steps depicted in the accompanying figures can be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

1. A device comprising:
a cap including a collection member configured to collect a biological sample and one or more members surrounding the collection member, the cap including one or more coupling members; and
a body including a reservoir configured to be filled with a solution and an information member, the information member at least demarcating an analysis region, the analysis region corresponding to a defined area of the body in which at a portion of the reservoir is visible, the body including one or more complimentary members configured to receive the coupling members of the cap and the reservoir being configured to receive the collection member and the one or more members surrounding the collection member.

2. The device according to claim 1, wherein the body includes a seal disposed above the reservoir and the one or more members surrounding the collection member are configured to break the seal.

3. The device according to claim 1, wherein the information member further includes identification information, the information member including one or more legends or scales representing one or more colors configured to correct an image of the analysis region for lighting conditions.

4. The device according to claim 1, wherein the body and the cap are configured to be mated and to be flush when mated.

5. The device according to claim 4, wherein the cap includes one or more tapered protruding members that surround the collection member.

6. The device according to claim 3, wherein the information member is disposed on an outer surface of the body.

7. The device according to claim 1, wherein the body is circular or rectangular.

8. The device according to claim 1, wherein the body is at least partially transparent.

9. The device according to claim 1, wherein the reservoir is an elongated channel.

10. The device according to claim 9, wherein the reservoir is disposed between the one or more coupling members.

11. A method of determining hemoglobin information for a collected biological sample from a diagnostic, screening or risk-assessment device, the device including an analysis region for a reservoir containing a pre-filled solution mixed with the biological sample and an information member including one or more scales, comprising:
processing a captured image of the analysis region and the device information to determine color information for the analysis region and one or more scales, the color information including a quantitative value representing one or more color attributes;
correcting the color information for the solution based on the color information for the scale; and
determining hemoglobin information based on the adjusted color information and stored profile information associated with the device.

12. The method according to claim 11, further comprising:
selecting the profile information from a plurality of stored profile information based on the device information, the profile information including a standardized scale for the solution.

13. The method according to claim 11, wherein the one or more color attributes include intensity, brightness, hue, among others, or a combination thereof.

14. The method according to claim 11, wherein the processing includes processing the image to determine proper orientation and distance based on one or more reference members provided on the device.

15. The method according to claim 11, wherein the determining the hemoglobin information includes correlating the adjusted color information to the stored profile information, the stored profile information including a three-dimensional plane associating color information associated with the solution and hemoglobin levels.

16. The method according to claim 11, further comprising:
displaying the hemoglobin information.

17. The method according to claim 11, wherein the collected biological sample is blood collected with a sample tube included with the device.

18. The method according to claim 11, wherein the hemoglobin information includes a hemoglobin value, a calculated or direct hematocrit value, and/or a disease state associated with the hemoglobin value and/or hematocrit value.

19. The method according to claim 11, wherein the one more scales correct the captured image for lighting conditions.

20. The method according to claim 11, further comprising:
causing a camera to capture an image of the device, the device including the analysis region and the information member; and
receiving the image of the device.
